(19) United States
(12) Patent Application Publication
(10) Pub. No.: US 2024/0269114 A1
(43) Pub. Date: Aug. 15, 2024

(54) Kit Comprising Quick-Release and Slow-Release Melatonin and Method of Using Same

(71) Applicant: Timeshifter, Inc., Southampton, NY (US)

(72) Inventor: Mickey Beyer-Clausen, Southampton, NY (US)

(73) Assignee: Timeshifter, Inc., Southampton, NY (US)

(21) Appl. No.: 18/440,681

(22) Filed: Feb. 13, 2024

Related U.S. Application Data
(60) Provisional application No. 63/484,565, filed on Feb. 13, 2023.

(51) Int. Cl. A61K 31/4045; A61J 7/04; G16H 20/10

(52) U.S. Cl. CPC A61K 31/4045; A61J 7/04; G16H 20/10

(57) ABSTRACT

A kit for effectuating a circadian shift in a user including a first plurality of melatonin doses, each being configured to deliver a first dosage of melatonin, and a second plurality of melatonin doses, each being configured to deliver a second dosage of melatonin that is not equal to the first dosage of melatonin. The kit may further include instructions for administering each of the first and second pluralities of melatonin doses.

### RELATED APPLICATIONS

This application claims priority under 35 U.S.C. § 119(e) of U.S. Provisional Patent Application Ser. No. (Attorney Docket No. 3788.00030) filed on and titled Kit Comprising Quick-Release and Slow-Release Melatonin and Method of Using Same. The content of this application is incorporated herein by reference.

### FIELD OF THE INVENTION

The present invention relates to a kit and methods for utilizing quick-release and slow-release versions of a variety of doses of melatonin for effectuating circadian rhythm shifts and facilitating sleep.

### BACKGROUND OF THE INVENTION

Misalignment of circadian rhythms is the result of a multitude of factors impacting various circadian clocks in an individual, in sometimes chaotic ways. There are many instances in which an individual may desire to change their circadian rhythms in a controlled manner, such as, for example, jet lag resulting from a change in time zone of the individual, adjusting to a new work schedule, needing to be at peak physical and/or mental performance at a certain time of day, and needing to maximize the efficacy of chronotherapeutics. Owing to the multitude of factors that can influence a circadian shift, a simplified mechanism that can be easily be prescribed/provided and easily understood, implemented, and complied with by the individual in order to facilitate a successful circadian shift is needed. Previous generic advice that relied on providing supplemental melatonin have provided single types of melatonin to the individual, i.e. recommended either a single type of quick-release or slow-release melatonin, with a range of low-dose and high-dose preparations. Moreover, these solutions did not provide guidance as to when to administer various dosages in response to a personal circadian shifting plan and did not recommend when to use slow-vs-fast release melatonin depending on the characteristics of the desired circadian shift or sleeping time. Accordingly, a kit that provides a variety of melatonin supplements and guidance for when to administer the same is desired.

### BRIEF DESCRIPTION OF THE DRAWINGS

FIG. 1 is a perspective view of a circadian rhythm shifting kit according to an embodiment of the invention.

FIG. 2 is a flowchart illustrating a method of administering two melatonin doses according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Additionally, although terms like “slow-release” and “quick-release” will be used in the application, all characterizations of release of materials are contemplated and included within the scope of the invention, including, but not limited to, controlled delivery, controlled release, delayed release, extended release, immediate release, long-acting, long-acting release, modified release, prolonged release, sustained action, sustained release, and timed release.

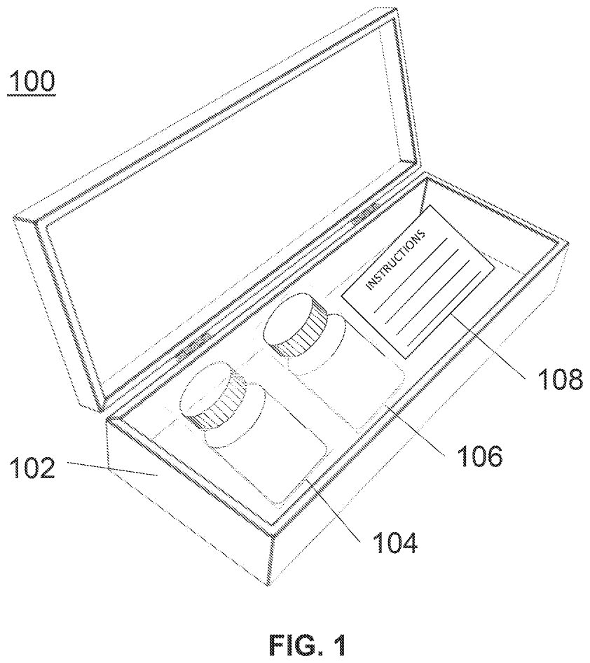

An embodiment of the invention, as shown and described by the various figures and accompanying text, provides a kit for effectuating a circadian shift in a user. Referring now to FIG. 1, a kit 100 according to an embodiment of the invention is presented. The kit 100 may comprise a container 102 having stored therein a first plurality of melatonin doses 104, a second plurality of melatonin doses 106, and instructions 108 for administration of the first and second pluralities of melatonin doses 104, 106. While melatonin doses are recited, any type of pharmaceutical delivery medium is contemplated and included within the scope of the invention, including, but not limited to, all oral dosage forms of pharmaceuticals, including pills, caplets, tablets, drinks, oral sprays, oral drops such as inhalers, chewing gum, lozenges, gummies and the like, with or without other substances. Other pharmaceutical delivery media contemplated by the invention include transdermal patches, nasal sprays, powders, creams or ointments, and any other delivery medium or method as is known in the art. It is contemplated and included within the scope of the invention that the first plurality of melatonin doses 104 may be in a first delivery method and packaging therefore and the second plurality of melatonin doses 106 may be in a second delivery method and packaging therefore, that may be the same as or different from the first delivery method. For example, the first delivery method may be a spray and the second delivery method may be a pill or capsule. As another example, the first delivery method may be a pill or capsule and the second delivery method may be a patch. These examples are exemplary only, do not limit the scope of the invention, and all possible combinations of delivery methods for the first and second delivery methods is contemplated and included within the scope of the invention.

The first and second pluralities of melatonin doses 104, 106 may be contained within at least one of a package and a delivery device. In pill, capsule, and other similar delivery media, the first and second pluralities of melatonin doses 104, 106 may be contained within at least one of a bottle, such as a bottle with a removable cap that may be configured to prevent a child from opening it, a blister pack, and the like.

Where the first and second pluralities of melatonin doses 104, 106 are chewing gum, transdermal patches, and the like, they may be individually wrapped and contained within a housing, such as a box, small container, and the like. In the case of chewing gum, there too the first and second pluralities of melatonin doses 104, 106 may be contained within a blister pack. In the case of a nasal spray, an oral spray, and all other types of sprays, the first and second pluralities of melatonin doses 104, 106 may be contained within a bottle having an appropriate spraying device attached thereto, as is known in the art. In some embodiments, the first and second melatonin doses may be contained within other consumable substances, such as bottles of potable fluids, including water, fruit juices, carbonated beverages, and the like.

As mentioned above, the first plurality of melatonin doses 104 may be configured to release the melatonin comprised thereby at a rate different from the second plurality of melatonin doses 106. In some embodiments, the first plurality of melatonin doses 104 may be an immediate-release dosage, configured to immediately deliver the melatonin comprised thereby into the bloodstream, and the second plurality of melatonin doses 106 may be a modified-release dosage, configured to deliver the melatonin at a delay after administration. In some embodiments, the first plurality of melatonin doses 104 may be a modified-release dosage configured to deliver its melatonin over a first time period and the second plurality of melatonin doses 106 may be a modified-release dosage configured to deliver its melatonin over a second time period that is greater than the first time period. In both such embodiments, the first plurality of melatonin doses 104 may be characterized as “quick-release” and the second plurality of melatonin doses 106 may be characterized as “slow-release”. Examples of the first time period may include delivering the melatonin over a period of sixty minutes, over a period of forty-five minutes, over a period of thirty minutes, over a period of fifteen minutes, over a period of ten minutes, over a period of five minutes, over a period of one minute, and any intermediate length of time. Furthermore, examples of the second time period may include delivering the melatonin over a period of sixty minutes, over a period of two hours, over a period of three hours, over a period of four hours, over a period of five hours, over a period of six hours, over a period of seven hours, over a period of eight hours, over a period of nine hours, over a period of eleven hours, over a period of twelve hours, over a period of twenty-four hours, and any intermediate length of time.

Additionally, in some embodiments, the first plurality of melatonin doses 104 may be configured to deliver a first dosage of melatonin and the second plurality of melatonin doses 106 may be configured to deliver a second dosage of melatonin. In some embodiments, the first dosage may be equal to the second dosage. In some embodiments, the first dosage may be less than the second dosage. In some embodiments, the first dosage may be greater than the second dosage. In some embodiments, the first dosage may be within a range from 0.05 milligrams (mg) to 20 milligrams. In some embodiments, the first dosage may be within a range from 0.1 mg to 10 mg. In some embodiments, the first dosage may be within a range from 0.05 mg to 5 mg. In some embodiments, the first dosage may be within a range from 0.05 mg to 3 mg. In some embodiments, the first dosage may be within a range from 0.05 mg to 1 mg. In some embodiments, the first dosage may be within a range from 0.1 mg to 1 mg. In some embodiments, the first dosage may be within a range from 0.2 mg to 1 mg. In some embodiments, the first dosage may be within a range from 0.3 mg to 1 mg. In some embodiments, the first dosage may be within a range from 0.4 mg to 1 mg. In some embodiments, the first dosage may be within a range from 0.5 mg to 1 mg. Further, all permutations of lower-and upper-bounds of the first dosage from 0.05 mg-20 mg are contemplated and included within the scope of the invention. In some embodiments, the second dosage may be within a range from 0.05 mg to 20 mg. In some embodiments, the second dosage may be within a range from 1 mg to 20 mg. In some embodiments, the second dosage may be within a range from 2 mg to 20 mg. In some embodiments, the second dosage may be within a range from 3 mg to 20 mg. In some embodiments, the second dosage may be within a range from 4 mg to 20 mg. In some embodiments, the second dosage may be within a range from 5 mg to 20 mg. In some embodiments, the second dosage may be within a range from 6 mg to 20 mg. In some embodiments, the second dosage may be within a range from 7 mg to 20 mg. In some embodiments, the second dosage may be within a range from 8 mg to 20 mg. In some embodiments, the second dosage may be within a range from 9 mg to 20 mg. In some embodiments, the second dosage may be within a range from 0.05 mg to 19 mg. In some embodiments, the second dosage may be within a range from 0.05 mg to 18 mg. In some embodiments, the second dosage may be within a range from 0.05 mg to 17 mg. In some embodiments, the second dosage may be within a range from 0.05 mg to 16 mg. In some embodiments, the second dosage may be within a range from 0.05 mg to 15 mg. In some embodiments, the second dosage may be within a range from 0.05 mg to 14 mg. In some embodiments, the second dosage may be within a range from 0.05 mg to 13 mg. In some embodiments, the second dosage may be within a range from 0.05 mg to 12 mg. In some embodiments, the second dosage may be within a range from 0.05 mg to 11 mg. Further, all permutations of lower-and upper-bounds of the second dosage from 0.05 mg-20 mg are contemplated and included within the scope of the invention.

The instructions 108 for administering the first and second pluralities of melatonin doses 104, 106 may include administration instructions for a variety of situations a user may desire to perform a circadian shift or to sleep when indicated. The instructions 108 may include administration instructions consistent with one or more of the following examples:

### Example 1

Jet lag, i.e. a mismatch of the user's internal circadian rhythm and the solar and social time of their present location after travel across multiple time zones, is mitigated by a circadian shift. Jet lag may be mitigated by administering a first dose, being a dose of the first plurality of melatonin doses 104 that is quick-release and low dosage, prior to and during travel. For example, the dose of the first plurality of melatonin doses 104 may be effective one of immediately and not more than one hour after administration and may be a dosage within a range from 0.05 mg to 20 mg of melatonin. Moreover, the dosage may be administered for several days before traveling, for example within an hour of sleep scheduled to begin circadian shifting before departure, or while in the act of traveling, such as while a passenger in a car, on an airplane, on a train, or other mode of transportation. A second dose may be administered after arrival at the destination, and for several days thereafter, for example within an hour of an intended time of going to sleep, the second dose being a dose of the second plurality of melatonin doses 106 that is slow-release and high dosage, compared to the rapidity of release and the dosage of the first dose. Specifically, the second dose may be within a range of 0.05 mg to 20 mg of melatonin and may be configured to be released after administration within a range from one hour to eight hours.

### Example 2

Shift work, i.e. a work schedule that does not conform to the typical night/day sleep cycle of humans, necessitates a change in the sleep schedule of an individual, and thus a user may desire to facilitate a change in their circadian rhythm. A first dose being a dose of the first plurality of melatonin doses 104 that is quick-release and low dosage may be administered prior to instantiation of a shift work schedule, e.g. prior to the user needing to work an evening or night shift. Such a first dose may be administered multiple times in days leading up to instantiation of a shift work schedule. In some embodiments, such a first dose may be administered the day of instantiation of a shift work schedule if a comparatively short amount of time is available for sleeping prior to the start of a shift work shift, for example, less than six hours. It is contemplated and included within the scope of the invention that the amount of time may vary depending on the length of the shift to be worked, the average length of time the user sleeps, how long since the user last had a complete sleep period, and how long since the user last had a shortened sleep period, e.g. a nap. A second dose being a dose of the second plurality of melatonin doses 106 that is slow-release and high dosage may be administered after completion of an individual shift work shift and/or when the user has the ability to sleep longer, for example for at least six hours, with no prescribed upper bound, so as to have a complete sleep episode. The length of time may again vary by individual and by the factors identified above. In some embodiments, additional melatonin doses 104 that are quick-release and low dosage or additional melatonin doses 106 that is slow-release and high dosage may be administered after completion of a series of shift work shifts (the end of a sequence of work shifts) to facilitate circadian re-adaptation to a normal day shift, an evening shift, a day of no work, a vacation, or any other shift type.

### Example 3

Increasingly, the efficacy of therapeutics, medical interventions as treatment for conditions, varies according to when they are administered according to the circadian phase of the patient's circadian rhythm(s) (central clock, peripheral clocks or combinations thereof). Accordingly, where such therapeutics are set to be administered at a particular time of day, for whatever reason, that is during a suboptimal phase of the patient's circadian rhythm(s), it may be desirous to shift the circadian rhythm(s) of the patient to optimize the efficacy of the therapy. There are two scenarios that may be addressed, namely, one where the therapy to be performed is a single instance within a reasonable time frame (e.g., once per week or month or longer) and will not be repeated in the near-term necessitating only a temporary shift in the patient's circadian cycle, and another where the therapy will be repeated more often (e.g., twice per week, three times a week, or more, or every day) and necessitates a persistent shift in the patient's circadian cycle. In the first scenario, a first dose being a dose of the first plurality of melatonin doses 104 or 106 may be administered prior to the therapy for several days at a time that is known to stimulate the desired shift , i.e. administered in the morning to delay the central clock if the therapy is to be performed in the afternoon or later than the individual's natural circadian phase, or administered in the evening if the therapy is to be performed in the morning or earlier than the individual's natural circadian phase , or administered in the evening if the therapy is to be performed in the evening.

The timing of these doses may be the same every day or may be changed systematically from day-to-day by 0.5-2 hours per day, depending on the individual's circadian phase or other indicators known to effect circadian phase (chronotype, age, sex, sleep timing, sleep duration, light/dark exposure), e.g., in the example of a shift to an earlier cycle (advance), an individual might receive the dose 104 or 106 in the three days before the therapy at 11 pm on a first day, 10 pm on a second day, and 9 pm on a third day. In the example of a shift to a later cycle (delay), an individual might receive the dose 104 in the three days before the therapy at 8 am on the first day, 9 am on the second day, and 10 am on the third day). The first dose may be a quick-release, low-dosage dose or slow-release high dosage dose. The dose type may change from day to day.

In the second scenario, a first dose may be administered that is similar to the first dose of the first scenario, i.e. a dose of the first plurality of melatonin doses 104 may be administered at a time during the day consistent with the therapy time of day. This first dose may be administered several times, on days preceding the therapy. A second dose being a dose of the second plurality of melatonin doses 106 being slow-release and high dosage may be administered in the evening, prior to a bedtime that coincides with or is shifted towards the circadian cycle that aligns with the future therapy. This second dose may facilitate maintaining the shifted sleep schedule on a persistent basis for the duration of the multiple therapies to be performed. Similarly, the second dose may be administered several times, once per day on the days leading up to the therapy.

### Example 4

Physical and/or mental performance has been documented as varying according to an individual's circadian rhythm. Accordingly, when there is a scheduled event or need to be at peak performance at a certain time of day, and that time of day coincides with a phase of the individuals' circadian cycle that is suboptimal, it may be desired to shift the circadian cycle of the individual such that the circadian phase associated with optimal performance is aligned as closely as possible with the scheduled event or otherwise identified time of day during which optimal performance is desired. The administration of first and second doses of the first and second pluralities of melatonin doses 104, 106 may be the same as in example 3, with differentiating between a temporary shift and a persistent shift, and the administration of only the first dose where a temporary shift is required and administration of first and second doses where a persistent shift is required. In some embodiments, a temporary shift may also include administration of the second dose. The shift may also begin several days before the day of peak performance and continue for several days after to readapt to normal circadian time after completion of the event.

### Example 5

Social jet lag, a phenomenon where the circadian rhythm of an individual user may shift during the weekend or other days when the individual does not work, is frequent in society. The typical pattern is the individual will tend to rise earlier and go to bed earlier on the day preceding a work day and during work days, and the individual will rise later and go to be later on days where they do not have to work the next day, although for some people, the opposite will be true. Such rapid and unfacilitated changes in the circadian cycle can have undesired consequences of the individual being tired at their first day back at work following one or more days of staying up late, or they may avoid activities they would otherwise engage in (e.g. social activities later in the evening) because of the circadian consequences of those activities. To avoid such undesired circadian shifts, the individual make administer a first dose being a dose of the first plurality of melatonin doses 104 that is quick-release and low dosage in the evening prior to going to sleep on the day prior to returning to work. The first dose may counter the undesired shift that might have caused the individual to stay awake later than desired. The individual may then administer a second dose being a dose of the second plurality of melatonin doses 106 in the evening on the day of returning to work, and during future work days, to facilitate maintaining the earlier circadian cycle established by the first dose. The melatonin doses 104 may be scheduled for several days before the first work day to preadapt circadian phase, and may be schedule for several days after the last work to facilitate shifting back to a desired phase. Similarly, the melatonin doses 106 may be continued after the last work day if an individual wishes to remain at their ‘work-day’ circadian phase during days off.

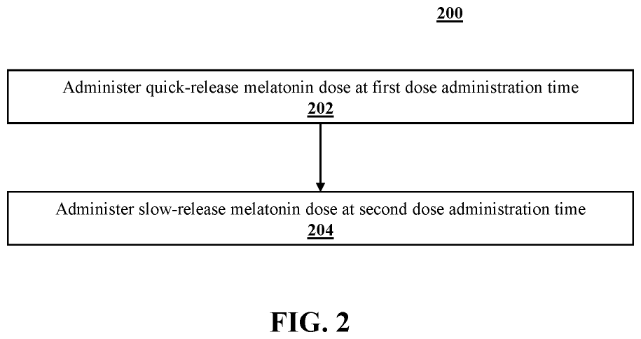

FIG. 2 is a flowchart of an example process 200. In some implementations, one or more process blocks of FIG. 2 may be performed by a device .

As shown in FIG. 2, process 200 may include administering a quick-release melatonin dose having a melatonin dosage at a first dose administration time (block 202). The melatonin dosage may be within a range from 0.5 mg to 1 mg. For example, a user or device may administer a quick-release melatonin dose having a melatonin dosage within a range from 0.5 mg to 1 mg at a first dose administration time, as described above. As also shown in FIG. 2, process 200 may include administering a slow-release melatonin dose at a second dose administration time subsequent to the first dose administration time (block 204). The slow-release melatonin dose may have a melatonin dosage within a range from 1 mg to 20 mg. For example, a user or device may administer a slow-release melatonin dose having a melatonin dosage within a range from 1 mg to 20 mg at a second dose administration time subsequent to the first dose administration time, as described above. The first and second times may be at times as described in Examples 1-5 above.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. For example, the shifting described above generally refers to shifting the central circadian clock, located in the suprachiasmatic nucleus (SCN), in order to shift rhythms controlled primarily by the central clock such as sleep, performance and mood. Other embodiments may involve shifting of other peripheral clocks that control metabolism, reproductive function, immune response or other circadian-controlled outcomes.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A kit for effectuating a circadian shift in a user comprising:
a first plurality of melatonin doses, each melatonin dose of the first plurality of melatonin doses being configured to deliver a first dosage of melatonin;
a second plurality of melatonin doses, each melatonin dose of the second plurality of melatonin doses being configured to deliver a second dosage of melatonin that is not equal to the first dosage of melatonin; and
instructions for administering each of the first and second pluralities of melatonin doses.

2. The kit of claim 1 wherein the first dosage is less than the second dosage.

3. The kit of claim 2 wherein:
the melatonin doses of the first plurality of melatonin doses are configured as quick-release doses; and
the melatonin doses of the second plurality of melatonin doses are configured as slow-release doses.

4. The kit of claim 3 wherein the instructions comprise:
instructions to administer a dose of the first plurality of melatonin doses during at least one of prior to travel and during travel; and
instructions to administer a dose of the second plurality of melatonin doses upon arrival at a travel location.

5. The kit of claim 3 wherein the instructions comprise:
instructions to administer at least one dose of the first plurality of melatonin doses prior to an instantiation of a shift work schedule; and
instructions to administer a dose of the second plurality of melatonin doses upon completion of a shift work shift of the shift work schedule.

6. The kit of claim 3 wherein the instructions comprise instructions to administer at least one dose of at least one of the first plurality of melatonin doses and the second plurality of melatonin doses prior to an administration of a therapy to the user.

7. The kit of claim 3 wherein the instructions comprise:
instructions to administer at least one dose of the first plurality of melatonin doses on days prior to administration of a therapy to the user; and
instructions to administer at least one dose of the second plurality of melatonin doses during an evening on the day before administration of the therapy to the user prior to a bedtime that at least one of coincides with and is shifted towards a circadian cycle that aligns with an optimal circadian cycle associated with the therapy.

8. The kit of claim 3 wherein the instructions comprise:
instructions to administer at least one dose of the first plurality of melatonin doses on days prior to a scheduled event having an associated optimal circadian cycle; and
instructions to administer at least one dose of the second plurality of melatonin doses in the evening on the day before the scheduled event prior to a bedtime that at least one of coincides with and is shifted towards a circadian cycle that aligns with the associated optimal circadian cycle.

9. The kit of claim 3 wherein the instructions comprise:
instructions to administer a dose of the first plurality of melatonin doses during an evening on a day prior to a return to work after at least one day of circadian rhythm-disrupting activity; and
instructions to administer a dose of the second plurality of melatonin doses during an evening on the day of returning to work.

10. The kit of claim 3 wherein:
the melatonin doses of the first plurality of melatonin doses are configured to release the melatonin comprised thereby over the course of not more than sixty minutes; and
the melatonin doses of the second plurality of melatonin doses are configured to release the melatonin comprised thereby over the course of not less than sixty minutes.

11. The kit of claim 2 wherein:
the first dosage is within a range from 0.05 mg to 1 mg; and
the second dosage is within a range from 1 mg to 20 mg.

12. The kit of claim 1 wherein:
the melatonin doses of the first plurality of melatonin doses are one of pills, caplets, tablets, drinks, oral sprays, oral drops such as inhalers, chewing gum, lozenges, gummies, transdermal patches, nasal sprays, powders, creams or ointments; and
the melatonin doses of the second plurality of melatonin doses are one of pills, caplets, tablets, drinks, oral sprays, oral drops such as inhalers, chewing gum, lozenges, gummies, transdermal patches, nasal sprays, powders, creams or ointments.

13. The kit of claim 1 wherein the melatonin doses of each of the first and second pluralities of melatonin doses are respectively contained within one of a package or a delivery device.

14. A kit for effectuating a circadian shift in a user comprising:
a first plurality of quick-release melatonin doses, each melatonin dose of the first plurality of quick-release melatonin doses being configured to deliver a first dosage of melatonin within a range from 0.05 mg to 1 mg;
a second plurality slow-release of melatonin doses, each melatonin dose of the second plurality of slow-release melatonin doses being configured to deliver a second dosage of melatonin within a range from 1 mg to 20 mg; and
instructions for administering each of the first and second pluralities of melatonin doses.

15. The kit of claim 14 wherein the instructions comprise at least one of:
a first instruction set comprising:
instructions to administer a dose of the first plurality of melatonin doses during at least one of prior to travel and during travel; and
instructions to administer a dose of the second plurality of melatonin doses upon arrival at a travel location;
a second instruction set comprising:
instructions to administer at least one dose of the first plurality of melatonin doses prior to an instantiation of a shift work schedule; and
instructions to administer a dose of the second plurality of melatonin doses upon completion of a shift work shift of the shift work schedule;
a third instruction set comprising instructions to administer at least one dose of at least one of the first plurality of melatonin doses and the second plurality of melatonin doses prior to an administration of a therapy to the user;
a fourth instruction set comprising:
instructions to administer at least one dose of the first plurality of melatonin doses on days prior to administration of a therapy to the user; and
instructions to administer at least one dose of the second plurality of melatonin doses during an evening on the day before administration of the therapy to the user prior to a bedtime that at least one of coincides with and is shifted towards a circadian cycle that aligns with an optimal circadian cycle associated with the therapy;
a fifth instruction set comprising:
instructions to administer at least one dose of the first plurality of melatonin doses on days prior to a scheduled event having an associated optimal circadian cycle; and
instructions to administer at least one dose of the second plurality of melatonin doses in the evening on the day before the scheduled event prior to a bedtime that at least one of coincides with and is shifted towards a circadian cycle that aligns with the associated optimal circadian cycle; and
a sixth instruction set comprising:
instructions to administer a dose of the first plurality of melatonin doses during an evening on a day prior to a return to work after at least one day of circadian rhythm-disrupting activity; and
instructions to administer a dose of the second plurality of melatonin doses during an evening on the day of returning to work.

16. The kit of claim 14 wherein:
the melatonin doses of the first plurality of melatonin doses are configured to release the melatonin comprised thereby over the course of not more than sixty minutes; and
the melatonin doses of the second plurality of melatonin doses are configured to release the melatonin comprised thereby over the course of not less than sixty minutes.

17. The kit of claim 14 wherein:
the melatonin doses of the first plurality of melatonin doses are one of pills, caplets, tablets, drinks, oral sprays, oral drops such as inhalers, chewing gum, lozenges, gummies, transdermal patches, nasal sprays, powders, creams or ointments;
and the melatonin doses of the second plurality of melatonin doses are one of pills, caplets, tablets, drinks, oral sprays, oral drops such as inhalers, chewing gum, lozenges, gummies, transdermal patches, nasal sprays, powders, creams or ointments.

18. The kit of claim 14 wherein the melatonin doses of each of the first and second pluralities of melatonin doses are respectively contained within one of a package or a delivery device.

19. A method of administering melatonin comprising:
administering a quick-release melatonin dose having a melatonin dosage within a range from 0.5 mg to 1 mg at a first dose administration time; and
administering a slow-release melatonin dose having a melatonin dosage within a range from 1 mg to 20 mg at a second dose administration time subsequent to the first dose administration time.

20. The method of claim 19 wherein the first and second dose administration times are respectively one of:
one of prior to travel and during travel, and upon arrival at a travel location;
prior to an instantiation of a shift work schedule and upon completion of a shift work shift of the shift work schedule;
on days prior to administration of a therapy to a user and during an evening on the day before administration of the therapy to the user prior to a bedtime that at least one of coincides with and is shifted towards a circadian cycle that aligns with an optimal circadian cycle associated with the therapy;
on days prior to a scheduled event having an associated optimal circadian cycle and in the evening on the day before the scheduled event prior to a bedtime that at least one of coincides with and is shifted towards a circadian cycle that aligns with the associated optimal circadian cycle; and
during an evening on a day prior to a return to work after at least one day of circadian rhythm-disrupting activity, and during an evening on the day of returning to work.
